(19) United States
(12) Patent Application Publication
(10) Pub. No.: US 2017/0147770 A1
(43) Pub. Date: May 25, 2017

(54) CRITICAL CARE PATIENT MONITORING SERVICE RECOMMENDATION USING DATA AND TEXT MINING TECHNIQUES

(71) Applicant: KONINKLIJKE PHILIPS N.V., EINDHOVEN (NL)

(72) Inventors: Derek Xu, Briarcliff Manor, NY (US); Eric Thomas Carlson, New York, NY (US); Oladimeji Feyisetan Farri, Ossining, NY (US); Lin Yang, Chandler, AZ (US); Larry James Eshelman, Ossining, NY (US)

(21) Appl. No.: 15/358,214

(22) Filed: Nov. 22, 2016

Related U.S. Application Data
(60) Provisional application No. 62/259,278, filed on Nov. 24, 2015.

(51) Int. Cl. G06F 19/00; A61B 5/00; A61B 5/0205

(52) U.S. Cl. CPC G06F 19/3406; A61B 5/02055; A61B 5/7275; A61B 5/742; G06F 19/322; A61B 5/024

(57) ABSTRACT

When monitoring patients in a general ward, clinical decision support risk scores are evaluated to determine whether a patient should be monitored using a spot check method whereby a caregiver periodically checks the patient, a continuous monitoring method whereby the patient is monitored by a monitoring device such as an electrocardiograph, or whether the patient requires transfer to a progressive care unit (PCU) or intensive care unit (ICU). When the number of patient monitors is not sufficient to assign a monitor to all patients for whom a monitor is desired, CDS score thresholds are adjusted to ensure that the neediest patients are assigned monitors.

### CROSS REFERENCE TO RELATED APPLICATIONS

The present application claims the benefit to U.S. Provisional Application Ser. No. 62/259,278, filed on Nov. 24, 2015. These applications are hereby incorporated by reference herein.

### BACKGROUND

Various embodiments find application in patient monitoring systems and methods. However, it will be appreciated that the described techniques may also find application in other patient assessment systems, other resource allocation methods, and the like. In hospital settings, patients receive the basic level of cares in the general ward and the highest level of cares in the intensive care unit (ICU). The general ward has a substantially large number of patient beds, relatively low nurse-to-patient ratio, and only provides spot check monitoring by nurses (nurses manually measure and record patients' vital signs) at intervals of, e.g., 4 hours or even less frequently. On the other hand, ICU has high nurse-to-patient ratio and provides both spot check and electronic monitoring services for every patient. In the general ward, deteriorating patients are transferred to higher level of care units such as ICU. This transfer action is usually decided by physicians, and is based on nurses' spot check measurements.

In clinical practice, some patients are neither stable enough to stay in general wards nor unstable enough to merit transfer to ICU. These patients are usually transferred to another special care unit: the progressive care unit (PCU). In PCU, each patient is monitored by both electronic monitors and spot check, as these patients need frequent monitoring to detect their deterioration events. In clinical practice, many patients fall into this category. As a result, PCU becomes crowded; and hospitals spend a lot on building new PCUs and on the costs in patient transfers between the general ward and PCU.

The present innovation provides new and improved systems and methods that facilitate providing a cost-effective solution to improving care quality in the general ward by reallocating hospital resources, thereby overcoming the above-referenced problems and others.

### SUMMARY

In accordance with one aspect, a method of managing allocation of patient monitoring devices and patient transfer among hospital wards comprises, via one or more processors, receiving spot check data that describes a level of stability of each of a plurality of patients being monitored periodically by one or more caregivers and electronic medical record (EMR) data associated with one or more of the plurality of patients, and evaluating the level of stability of each of the plurality of patients. The method further comprises determining whether one or more of the plurality of patients' needs one of continuous monitoring or transfer to an intensive care unit (ICU) or progressive care unit (PCU) as a function of the patient's level of stability.

According to another aspect, a method for managing allocation of patient monitoring devices and patient transfer among hospital wards comprises, via one or more processors, receiving spot check data that describes a level of stability of each of a plurality of patients being monitored periodically by one or more caregivers and electronic medical record (EMR) data associated with one or more of the plurality of patients, evaluating the level of stability of each of the plurality of patients, determining whether one or more of the plurality of patients' needs one of continuous monitoring or transfer to an intensive care unit (ICU) or progressive care unit (PCU) as a function of the patient's level of stability. The method further comprises receiving patient vital sign information for one or more of the plurality of patients; using a clinical decision support (CDS) technique to calculate a risk score for one or more patients based on the vital sign information and EMR data, and, for each patient, transmitting a monitoring recommendation, which is based on the patient's calculated risk score, to a caregiver via a graphical user interface.

According to another aspect, a system that facilitates managing allocation of patient monitoring devices and patient transfer among hospital wards comprises a processor configured to evaluate spot check data that describes a level of stability of each of a plurality of patients being monitored periodically by one or more caregivers, and electronic medical record (EMR) data associated with one or more of the plurality of patients, evaluate the level of stability of each of the plurality of patients, and determine whether one or more of the plurality of patients' needs one of continuous monitoring or transfer to an intensive care unit (ICU) or progressive care unit (PCU) as a function of the patient's level of stability. The system can also make a recommendation to reduce the monitoring level for patients who are already on continuous monitoring. This feature saves monitoring resources for other patients and can also improve patients' experience.

Further advantages of the subject innovation will be appreciated by those of ordinary skill in the art upon reading and understand the following detailed description.

### BRIEF DESCRIPTION OF THE DRAWINGS

The drawings are only for purposes of illustrating various aspects and are not to be construed as limiting.

FIG. 1 illustrates a flow chart for automatically determining a monitoring approach for monitoring a patient or transferring a patient to a different ward, in accordance with one or more features described herein.

FIG. 2 illustrates a flow chart for using a clinical decision support (CDS) approach to assess patients' conditions, in accordance with one or more features described herein.

FIG. 3 illustrates a flow chart for employing an NLP engine, as well as text mining and data mining algorithms to assess patients' conditions, in accordance with one or more feature described herein.

FIG. 4 illustrates a system that facilitates automatically determining a patient monitoring approach based on monitored patient vital signs, clinical data, and the like, according to one or more features described herein.

### DETAILED DESCRIPTION

The herein-described systems and methods overcome the aforementioned problems by using vital signs (e.g., heart rate, respiratory rate, etc.), electronic medical records (EMR), and clinical notes to determine which monitoring approach should be used (e.g. spot check or continuous monitoring). Patients' conditions are assessed by two sets of algorithms: text mining algorithms that extract and summarize information from clinical notes; and clinical decision support (CDS) algorithms that take as input patients' vital sign data and EMR data, as well as information extracted from the clinical notes by text mining, and output risk scores. In this manner, one of three major responses is determined based on patients' conditions: monitor patient(s) by spot check approach; monitor patient(s) by continuous monitoring approach; or transfer patient(s) to ICU or PCU.

The described systems and methods can be applied in, e.g., the low acuity level of care units in hospitals: e.g. the general ward, medical/surgical room, observation unit, or post-anesthesia care unit (PACU). In these care units, the described systems and methods can improve the care quality and reduce the overall cost during patients' stay. The described innovation can also be employed in systems (e.g. EMR, IntelliSpace Critical Care and Anesthesia (ICCA) or eCareManager) to create alerts and recommendations, as well as in hospital systems, remote telehealth solutions, hospital to home applications, or growth markets.

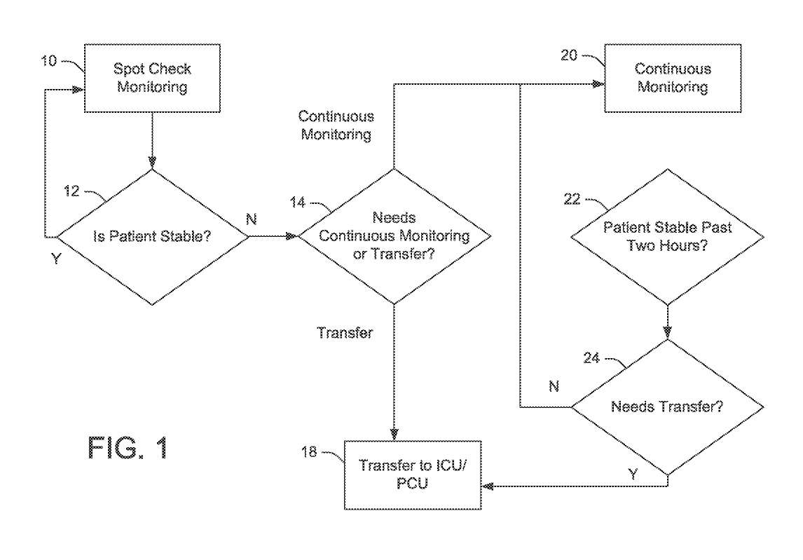

FIG. 1 illustrates a flow chart for automatically determining a monitoring approach for monitoring a patient or transferring a patient to a different ward, in accordance with one or more features described herein. At 10, spot check monitoring is initiated for patients in the general wards. Spot check data describes a level of stability of each of a plurality of patients being monitored periodically by one or more caregivers The patients' conditions are assessed from the spot check data and EMR data. At 12 a determination is made regarding whether the patient is stable (i.e., patient instability is below a predetermined patient stability threshold). If so, the method reverts to 10 for continued spot check monitoring. If the patient is unstable, then at 14, a determination is made regarding whether the patient needs either continuous monitoring or transferring to the ICU or PCU, taking into account the level of confidence (e.g., determined by the amount of available data and the statistical distribution of the data) in the patient's condition as well as the level in instability. For example, if there is indication that the patient is unstable, but the level of confidence is low, the determination at 14 recommends continuous monitoring, at 16. If the patient is unstable and the confidence is high, then the determination at 14 recommends transfer to one of the PCU or ICU, at 18. For patients being continuously monitored in the general wards, the proposed algorithm will take the vital sign data from the continuous monitors, update the patient's conditions, and determine the responses in a frequent manner (e.g., every 10 minutes, every 5 minutes, every 30 minutes, etc.).

At 20, a determination is made regarding whether the patient has been stable for a predetermined time period (e.g., 2 hours, 1 hour, or some other predetermined time period). If the patient has been stable for a specified period of time, e.g., 2 hours, the patient is returned to spot check monitoring, at 10, or a less-frequent measurement of certain vital signs. If the patient has not been stable, a determination is made regarding whether the patient requires transfer to the PCU or ICU, at 22. If transfer is not required, the method reverts to 20 for continued continuous monitoring. If the patient's condition indicates the patient is unstable and needs transfer, the patient is transferred to ICU or PCU, at 24.

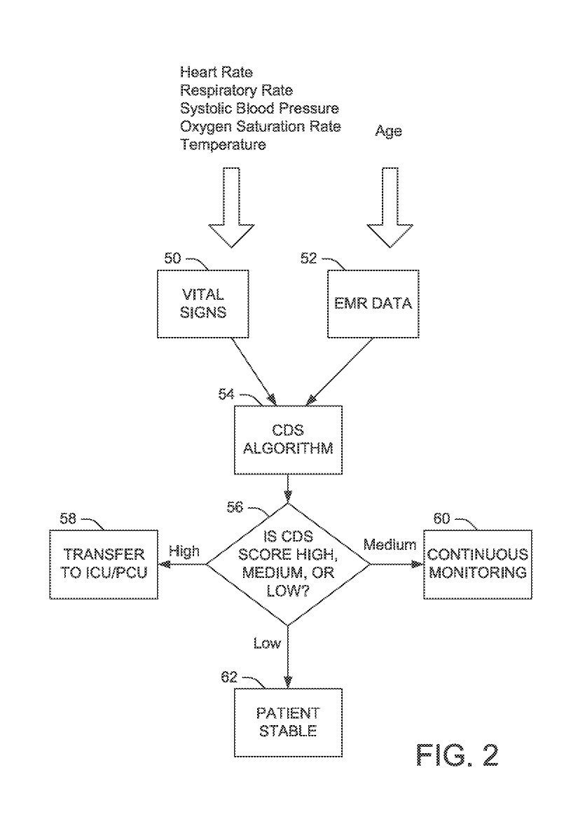

FIG. 2 illustrates a flow chart for using a clinical decision support (CDS) approach to assess patients' conditions, in accordance with one or more features described herein. At 50, patient vital sign information (e.g., heart rate, respiratory rate, systolic blood pressure, oxygen saturation rate, temperature, etc.) are monitored and stored. At 52, electronic medical record (EMR) data is stored. At 54, CDS algorithms are used to assess patients' conditions based on the stored vital sign data and EMR data, and to trigger the alerts to caregivers. Once risk scores are calculated from CDS algorithms, the patients' conditions are assessed at 56 to determine whether the risk scores were high, median, or low (e.g., above or below one or more thresholds). Additionally, a confidence level associated with the risk score is calculated and evaluated. The high, median, or low scores can be determined by machine learning techniques that train the scores from retrospective data. If the patient score is determined to be high (e.g., above an upper threshold) at 56, then at 58 an alert is triggered to inform a caregiver that the patient should be transferred to the PCU or ICU. If the patient score is determined to be medium (e.g., below the upper threshold but below a lower threshold) at 56, then an alert is triggered to inform a caregiver that the patient should be continuously monitored, at 60. If the patient score is determined to be low (e.g., at or below a lower threshold) at 56, then an alert is triggered to inform a caregiver that the patient is stable, at 62. The patient monitor recommendations are determined based on the patients' conditions.

According to an example, the first threshold is set to e.g., 50, so that patients having a score of 50-100 are recommended for transfer to a PCU or ICU. The second threshold is set to e.g., 30, so that patients having a score of 30-50 are recommended for continuous monitoring. Patients having a score below 30 are recommended for periodic spot checking by a caregiver. The first and second thresholds can also be adjusted so that when only a small number of monitor are available for a given type of condition, a corresponding number of patients falls between the first and second thresholds while the rest are recommended for transfer or spot check monitoring.

In one embodiment, the patients' CDS scores are calculated and evaluated periodically (e.g., every 5 minutes, every 20 minutes, etc.). In another embodiment, the CDS scores are calculated and evaluated continuously. When M monitors are available for a given disease group, the CDS scores of all patients in the disease group are evaluated to identify M patients whose scores are below the first (upper) risk score threshold and so do not need to be transferred, but who are in need of continuous monitoring (CDS scores above the second (lower) lower. For instance, using the above example, if M=8, and there are 10 patients above the second threshold and below the first threshold, 8 of whom have a CDS score between 35 and 50, then the second threshold can be raised to 35 to weed out the 2 least needy patients.

In this manner, the described methods and systems facilitate providing continuous monitoring of patients using relatively few monitors. For instance, when it is not feasible to assign an EKG monitor to every patient in a ward, patients' stability trends are evaluated and recommendations are generated to assign monitors to the neediest patients for continuous monitoring. The type of monitor assigned depends on the monitors available and the condition being monitored.

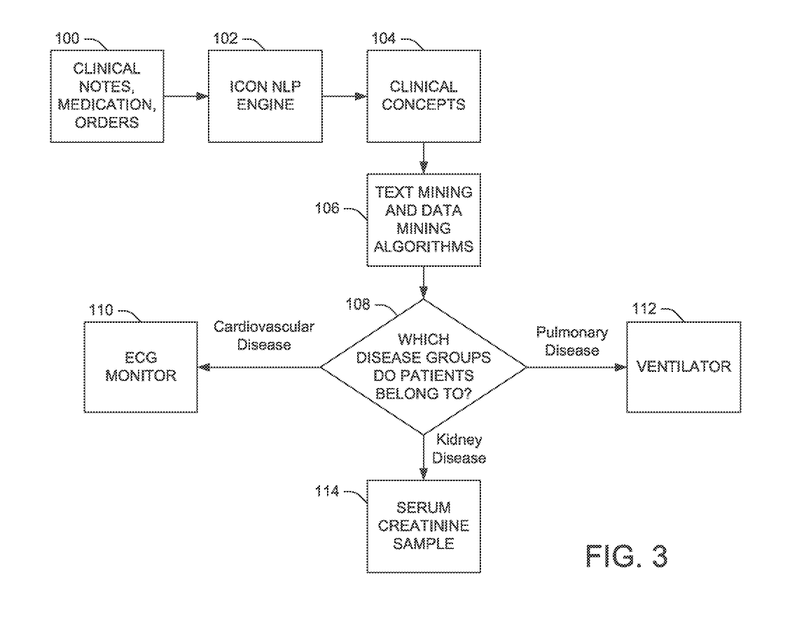

FIG. 3 illustrates a flow chart for employing an NLP engine, as well as text mining and data mining algorithms to assess patients' conditions, in accordance with one or more feature described herein. Clinical notes can also be used to provide information for the monitoring recommendation. Clinical notes are free-text based documents in which are recorded patients' findings and conditions along their care continuum. These notes contain information that is relevant for disease prediction. The patient monitoring recommendation can be provided based on patients' diseases that are summarized from these notes. For instance, EKG monitors can be recommended for cardiovascular patients. In clinical practice, due to the typically vast number of clinical notes per patient, physicians are not able to collect all of a given patent's clinical notes to make fully informed decisions. By automating the collection and analysis of a patient's clinical notes, physician decision-making confidence is improved.

At 100, clinical notes, patient medication orders, and the like are input into natural language processing (NLP) engine 102 engine (e.g. Philips ICON (Information extraction using Clinical Ontologies) NLP technology pipeline or the like) that is employed to extract clinical concepts 104 from the clinical notes 100. The clinical concepts are then analyzed by text mining and data mining algorithms 106. Patients are then classified into various disease groups at 108, such as e.g. cardiovascular disease, pulmonary disease, liver disease, kidney disease, etc. Monitor recommendations are established based on those disease group classifications. For instance, if the patient is classified as having cardiovascular disease, an EKG monitor is recommended, at 110. If the patient is classified as having pulmonary disease, a respiratory monitor is recommended, at 112. If the patient is classified as having kidney disease, a respiratory monitor is recommended, at 114.

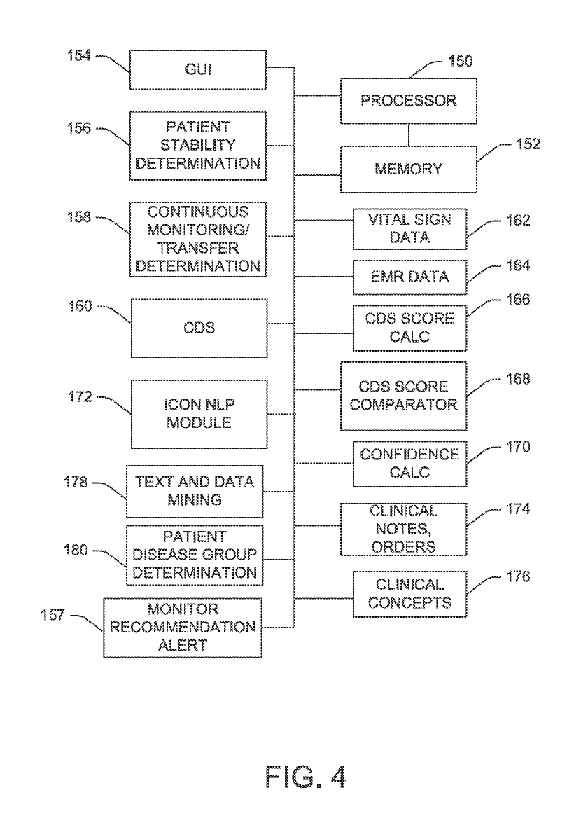

FIG. 4 illustrates a system that facilitates automatically determining a patient monitoring approach based on monitored patient vital signs, clinical data, and the like, according to one or more features described herein. The system includes a processor 150 that executes, and a memory that stores, one or more computer-executable modules for performing the various functions, methods, etc., described herein. “Module,” as used herein, denotes a computer-executable algorithm, routine, application, program, or the like, and/or a processor that executes said computer-executable algorithm, routine, application, program, or the like.

It will be understood that the processor 150 executes, and the memory 152 stores, computer executable instructions for carrying out the various functions and/or methods described herein. The memory 152 may be a computer-readable medium on which a control program is stored, such as a disk, hard drive, or the like. Common forms of computer-readable media include, for example, floppy disks, flexible disks, hard disks, magnetic tape, or any other magnetic storage medium, CD-ROM, DVD, or any other optical medium, RAM, ROM, PROM, EPROM, FLASH-EPROM, variants thereof, other memory chip or cartridge, or any other tangible medium from which the processor 150 can read and execute. In this context, the described systems may be implemented on or as one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphics processing unit (GPU), or PAL, or the like.

The system further comprises a graphical user interface (GUI) 154 via which monitoring and/or patient transfer alerts and recommendations are delivered to patients. Once spot check monitoring has been initiated for patients in a general ward, the patients' conditions are automatically assessed by a patient stability determination module 156 that evaluates spot check data (e.g., stored in memory 152 upon entry by a caregiver) and EMR data. If so, on a per-patient basis, an alert or other indication is generated by a monitor recommendation alert module 157 and provided to the caregiver(s) via the GUI that the patient is approved to receive continued spot check monitoring. If the patient is unstable, determined to be unstable by the patient stability determination module 156, a determination is made by a continuous monitoring/patient transfer determination module regarding whether the patient needs either continuous monitoring (e.g., via a patient monitor such as an EKG monitor, a respiratory monitor, frequent blood sample analysis monitoring, or the like) or transferring to the ICU or PCU, taking into account the level of confidence in the patient's condition as well as the level in instability. For example, if there is indication that the patient is unstable, but the level of confidence is low, the continuous monitoring/patient transfer module 158 recommends continuous monitoring. If the patient is unstable and the confidence is high, then the continuous monitoring/patient transfer module 158 recommends transfer to one of the PCU or ICU. For patients being continuously monitored in the general wards, the continuous monitoring/patient transfer module 158 evaluates vital sign data from the continuous monitors, update the patient's conditions, and determines the responses in a frequent manner (e.g., every 10 minutes, every 5 minutes, every 30 minutes, etc.).

The patient stability determination module also determines a duration of patient stability, i.e., whether the patient has been stable for a predetermined time period (e.g., N hours, where N is an integer). If the patient has been stable for the specified period of time, e.g., 2 hours, the continuous monitoring/patient transfer module 158 transmits a recommendation or alert to the GUI that the patient can be returned to spot check monitoring. If the patient has not been stable for the predetermined time period, the continuous monitoring/patient transfer module 158 determines whether the patient requires transfer to the PCU or ICU. If transfer is not required, continuous monitoring is continued. If the patient's condition indicates the patient is unstable and needs transfer, the continuous monitoring/patient transfer module 158 sends an alert or recommendation to the GUI indicating that the patient should be transferred to ICU or PCU.

The system also includes a clinical decision support (CDS) module 160 that evaluates stored patient vital sign information 162 (e.g., heart rate, respiratory rate, systolic blood pressure, oxygen saturation rate, etc.) and electronic medical record (EMR) data 164. The CDS module assesses patients' conditions based on the stored vital sign data and EMR data, and triggers the continuous monitoring/patient transfer module 158 to send an appropriate alert to caregivers via the GUI. A CDS score calculator module 166 calculates CDS risk scores and a CDS score comparator module 166 evaluates the risk scores to determine whether the risk scores were high, median, or low (e.g., above or below one or more thresholds). Additionally, a confidence level calculator module 170 calculates a level of confidence associated with the risk score is calculated and evaluated. The high, median, or low scores can be determined by machine learning techniques that train the scores from retrospective data. If the patient score is determined to be high (e.g., above an upper threshold) an alert is triggered to inform a caregiver that the patient should be transferred to the PCU or ICU. If the patient score is determined to be medium (e.g., below the upper threshold but below a lower threshold) an alert is triggered to inform a caregiver that the patient should be continuously monitored. If the patient score is determined to be low (e.g., at or below a lower threshold) an alert is triggered to inform a caregiver that the patient is stable. The patient monitor recommendations are determined based on the patients' conditions.

The system further includes an NLP module 172, which evaluates clinical notes and medicinal orders 174 to extract clinical concepts 176 (e.g. diagnoses, symptoms, procedures, medications, etc.) therefrom. The Clinical concepts are evaluated by a text and data mining module 178 to assess patients' conditions. Clinical notes can also be used to provide information for the monitoring recommendation provided by the continuous monitoring/patient transfer module 158. Patients are then classified into various disease groups, such as e.g. cardiovascular disease, pulmonary disease, and kidney disease, etc. Monitor recommendations are established by the continuous monitoring/patient transfer module 158 based on those disease group classifications. For instance, if the patient is classified as having cardiovascular disease, an EKG monitor is recommended. If the patient is classified as having pulmonary disease, a respiratory monitor is recommended. If the patient is classified as having kidney disease, a respiratory monitor is recommended.

The innovation has been described with reference to several embodiments. Modifications and alterations may occur to others upon reading and understanding the preceding detailed description. It is intended that the innovation be construed as including all such modifications and alterations insofar as they come within the scope of the appended claims or the equivalents thereof

We claim:

1. A method of managing allocation of patient monitoring devices and patient transfer among hospital wards, comprising:
via one or more processors:
receiving spot check data that describes a level of stability of each of a plurality of patients being monitored periodically by one or more caregivers and electronic medical record (EMR) data associated with one or more of the plurality of patients;
evaluating the level of stability of each of the plurality of patients;
determining whether one or more of the plurality of patients' needs one of continuous monitoring or transfer to an intensive care unit (ICU) or progressive care unit (PCU) as a function of the patient's level of stability.

2. The method according to claim 1, wherein upon a determination that the instability of the given patient is below a predetermined patient stability threshold, and a confidence level in the patient stability determination is below a predetermined confidence level, signaling a graphical user interface (GUI) to display an alert to a caregiver assign a monitoring device to the given patient for automated continuous monitoring.

3. The method according to claim 1, wherein upon a determination that the instability of the given patient is below a predetermined patient stability threshold, and a confidence level in the patient stability determination is above a predetermined confidence level, signaling a graphical user interface (GUI) to display an alert to a caregiver to transfer the given patient to one of the PCU and the ICU.

4. The method according to claim 1, further comprising:
determining whether the patient has been stable for a predetermined time period.

5. The method according to claim 4, wherein if the patient is determined to have been stable for the predetermined time period, further comprising providing an alert to return the patient to a spot check monitoring protocol.

6. The method according to claim 4, wherein if the patient is determined not to have been stable for the predetermined time period, further comprising determining whether the patient requires transfer to the PCU or ICU.

7. The method according to claim 6, wherein if patient transfer is not determined to be required, further comprising providing an alert message that continuous monitoring is to be continued for the patient.

8. The method according to claim 6, wherein if patient transfer is determined to be required, further comprising providing an alert message the patient is to be transferred to one of a PCU and an ICU.

9. The method according to claim 4, wherein the predetermined time period is on the order of N hours, where N is an integer.

10. A method of managing allocation of patient monitoring devices and patient transfer among hospital wards, comprising:
via one or more processors:
receiving spot check data that describes a level of stability of each of a plurality of patients being monitored periodically by one or more caregivers and electronic medical record (EMR) data associated with one or more of the plurality of patients;
evaluating the level of stability of each of the plurality of patients;
determining whether one or more of the plurality of patients' needs one of continuous monitoring or transfer to an intensive care unit (ICU) or progressive care unit (PCU) as a function of the patient's level of stability.
receiving patient vital sign information for one or more of the plurality of patients;
using a clinical decision support (CDS) technique to calculate a risk score for one or more patients based on the vital sign information and EMR data; and
for each patient, transmitting a monitoring recommendation, which is based on the patient's calculated risk score, to a caregiver via a graphical user interface.

11. The method according to claim 10, wherein the vital sign information comprises one or more of: heart rate, respiratory rate, systolic blood pressure, oxygen saturation rate, and temperature.

12. The method according to claim 10, further comprising:
comparing a calculated risk score for each of the plurality of patients to a first risk score threshold to determine whether the risk score is high or moderate; and
comparing a calculated risk score for each of the plurality of patients to a second risk score threshold to determine whether the risk score is moderate or low.

13. The method according to claim 12, further comprising:
for each high risk patient, transmitting a monitoring recommendation recommending transfer to one of a PCU and an ICU;
for each moderate risk patient, transmitting a monitoring recommendation recommending continuous monitoring and assigning a monitoring device; and
transmitting a monitoring recommendation for each low risk patient, recommending spot check monitoring.

14. The method according to claim 10, further comprising:
inputting one or more of clinical notes and patient medication orders (100) into a natural language processing (NLP) engine;
extracting clinical concepts from the clinical notes;
analyzing the clinical concepts by performing text mining and data mining; and
classifying patients into a plurality of disease groups for which patient monitors are available for continuous monitoring.

15. The method according to claim 14, wherein the disease groups comprise cardiovascular disease, pulmonary disease, kidney disease, and liver disease.

16. The method according to claim 14, further comprising:
assigning patient monitors to patients as a function of patient disease group and CDS score such that, within each disease group, patients with higher CDS scores are assigned monitors with priority over patients with lower CDS scores.

17. The method according to claim 16, further comprising:
when M patient monitors are available for a give disease group, adjusting the first and second risk score thresholds to ensure that the M patient monitors are assigned to M patients having the M highest CDS score below the first risk score threshold, where M is an integer.

18. A system that facilitates managing allocation of patient monitoring devices and patient transfer among hospital wards, comprising:
a processor configured to:
evaluate spot check data that describes a level of stability of each of a plurality of patients being monitored periodically by one or more caregivers, and electronic medical record (EMR) data associated with one or more of the plurality of patients;
evaluate the level of stability of each of the plurality of patients; and
determine whether one or more of the plurality of patients' needs one of continuous monitoring or transfer to an intensive care unit (ICU) or progressive care unit (PCU) as a function of the patient's level of stability.

19. The system according to claim 18, wherein the processor is further configured to receive patient vital sign information for one or more of the plurality of patients, and further comprising:
a clinical decision support (CDS) module that calculates a risk score for one or more patients based on the vital sign information and EMR data;
wherein the processor is further configured to, for each patient, transmit a monitoring recommendation, which is based on the patient's calculated risk score, to a caregiver via a graphical user interface.

20. The system according to claim 19, further comprising:
a natural language processing (NLP) engine that evaluates that one or more of clinical notes and patient medication orders, and extracts clinical concepts from the clinical notes;
wherein the processor is further configured to:
analyze the clinical concepts by performing text mining and data mining;
classify patients into a plurality of disease groups for which patient monitors are available for continuous monitoring;
and assign available patient monitors to one or more patients in each disease group as a function of the one or more patients' risk score.
